(19) United States
(12) Patent Application Publication
(10) Pub. No.: US 2011/0172495 A1
(43) Pub. Date: Jul. 14, 2011

(54) SURGICAL RETRACTOR

(76) Inventor: David N. Armstrong, Atlanta, GA (US)

(21) Appl. No.: 13/069,783

(22) Filed: Mar. 23, 2011

Related U.S. Application Data
(60) Provisional application No. 61/341,100, filed on Mar. 26, 2010.

(51) Int. Cl. A61B 1/32

(52) U.S. Cl. 600/233

(57) ABSTRACT

A biocompatible deformable retractor provides desired advanced organ retaining during surgical procedures. The retractor is generally self-retaining once deployed and provides surgical field without the interference from the retained organs. The deployed retractor may adopt convex, planar, or concave configurations inside a body cavity. The retractor comprises a deformable resilient frame and a deformable membrane, is approximately planar or a non-planar convex shape in its natural un-deformed configuration. The membrane can be transparent to provide additional viewing capability of the retained organs.

### CROSS REFERENCE TO RELATED APPLICATIONS

This application claims priority to U.S. provisional patent application Ser. No. 61/341,100, filed on Mar. 26, 2010 to Armstrong, entitled “3-Dimensional Laparoscopic Retractor,” incorporated herein by reference.

### FIELD OF THE INVENTION

The inventions, in general, are related to retractors for retaining organ to provide working space during surgery, especially laparoscopic procedures. The inventions are further related to method of using and making such retractors.

### BACKGROUND

One of the greatest technical challenges during hand-assisted laparoscopic surgery such as sigmoid colectomy or ileo-colic resection is retracting loops of small bowel which drop repeatedly into the surgical field and obscure visualization. The usual method to prevent this is positioning the operating table into steep Trendelenberg position, but this risks patient injury and positional hypotension. Devices to prevent prolapse of small bowel loops into the operative field during laparoscopic or open abdomino-pelvic surgery have been developed. For example, small bowel is known to be packed proximally in the abdomen using laparotomy pads or towels. Laparotomy pads or towels inadvertently left inside the abdomen, however, pose the risk of surgical complication with serious consequences. Other methods of retracting the small bowel into the upper abdomen utilize a plastic bag, into which the small bowel is placed and usually secured by means of a pull-cord around the opening of the bag. This approach does not provide retraction of the small bowel out of the pelvis and additionally risks strangulation of the bowel itself from constriction of the mesenteric blood supply.

### SUMMARY OF THE INVENTION

In a first aspect, the invention pertains to a biocompatible deformable retractor comprising a deformable resilient frame and a deformable membrane. The frame comprises a border around a majority or all of a perimeter around a central opening with the membrane secured around at least a portion of the border to form a restraining structure across the central opening. The retractor is approximately planar or a non-planar convex shape in its natural un-deformed configuration. The retractor can have a round, square, oval, rectangular, oblong, or polyhedral shape. Alternatively, the retractor can have a composite shape such as bean, figure of eight, or U-shape. The various shaped retractors in a natural un-deformed configuration can have a perimeter of approximately 5 inches to 200 inches (12.7 cm to 508 cm). The frame has across section through the frame, which can be can be round, oval, semi-circular, triangular, or polygonal having a perimeter from about 0.01 inch (0.25 cm) to about 3.94 inches (10 cm). The frame can be made, for example, from polymer, metal, or a combination thereof. In some embodiments, it may be desirable to have a cushion around the frame. The membrane can be made from a biocompatible sheet, fabric, net, or a combination thereof. In some embodiments, the membrane is transparent. In some embodiments, the retractor may have perforation through the membrane. The retractor in general can be self-extending.

In another aspect, the invention pertains to a method of using a retractor to retract one or more organs in a body cavity to form a working space in a living subject during a surgical procedure. The retractor can be deployed inside the body cavity by deforming the retractor, inserting the deformed retractor into the body cavity through a surgical opening, and releasing the retractor in the body cavity to retract the organ in the cavity to form the working space. Once deployed, the retractor can be self-retaining inside the cavity with the frame pushes against the wall of the cavity while the membrane presses against the organ to form the working space inside the cavity. In some embodiments, the deployed retractor has a convex shape pushing against the organ to make the working space. When a bean shaped retractor is deployed, the concavity of the bean shape can be placed over retroperitoneal vessel and/or conduit to prevent compression or occlusion of the vessel and/or conduit.

The surgical procedure can then be performed with assistance provided by the working space. In some embodiments, at least a portion of the membrane of the retractor remains attached to the frame throughout the procedure. During the procedure, the retractor may be repositioned to create different working spaces. Additionally, the membrane of the retractor may be punctured by laparoscopic instruments or laparoscopes if desired. After the surgical procedure, the retractor can be removed from the body cavity through the surgical opening. In one embodiment, the organ is GI tract, the body cavity is peritoneal cavity, and the working space is pelvic cavity. The surgical procedure may be a laparoscopic surgery or an open surgery. It may be a surgery of the GI tract, urinary system, reproductive system, abdominal wall, or pelvic floor. In some embodiments, the surgical procedure is open laparotomy, mini-laparotomy, pure laparoscopic surgery, hand assisted laparoscopic surgery, or single incision laparoscopic surgery.

In a further aspect, the invention pertains to method of making a biocompatible deformable retractor. The method comprises securing a membrane to a biocompatible deformable resilient frame to form the retractor. In some embodiments, the frame is approximately planar or a non-planar convex shape in its natural un-deformed configuration. The frame generally forms a border around a majority or all of the perimeter of a central opening, and the membrane is secured around at least a portion of the border to form a restraining structure within the central opening. The retractor may be further attached to a wound retractor or a laparoscopic access device.

### BRIEF DESCRIPTION OF THE DRAWINGS

FIG. 1a is a top plan view of a circular retractor with a deformable resilient frame and a transparent membrane secured around the frame.

FIG. 1b is a schematic diagram of a vertical cross sectional view along the b-b line of the retractor of FIG. 1a showing round cross section of the frame with wrap around membrane, in which the vertical reference is relative to a horizontally oriented retractor frame.

FIG. 1c shows schematic diagrams of alternative cross section shapes of the frame.

FIG. 2a is a schematic diagram of the circular retractor of FIG. 1a showing membrane secured around the frame with an adhesive.

FIG. 2b is a schematic diagram of the circular retractor of FIG. 1a showing membrane secured around the frame with stitches.

FIG. 2c is a schematic diagram of the circular retractor of FIG. 1a showing membrane secured around the frame with staples

FIG. 2d is a schematic diagram of a round shape retractor having perforations on the membrane.

FIGS. 3a-f are schematic diagrams of planar retractors of square, oval, rectangle, oblong, bean, “U” or horse shoe, or shape.

FIG. 3g is a schematic diagram of a retractor with a natural non-planar convex shape.

FIG. 4a is a photograph of the retractor of FIG. 1a twisted into a figure of 8 configuration.

FIG. 4b is a photograph of the retractor of FIG. 4a, folded into a two layer deformed configuration.

FIG. 4c is a schematic diagram of the retractor of FIG. 1a folded into a three consecutive oval shape.

FIG. 5a is a schematic diagram of peritoneal cavity showing viscera.

FIG. 5b is a schematic diagram of peritoneal cavity of FIG. 5a with a retractor deployed in a convex shape inside the cavity making a working space.

FIG. 6 is a photograph of the folded retractor of FIG. 4b being inserted through a wound access device into a body cavity of a patient during surgery.

FIG. 7 is a photograph of the retractor of FIG. 6 being deployed in the body cavity showing the organ being retained by the retractor through the transparent membrane.

FIG. 8 is a photograph of the working space created by the deployed retractor of FIG. 7 in the body cavity.

### DETAILED DESCRIPTION OF PREFERRED EMBODIMENTS

The improved retractors in general have a deformable resilient frame and a membrane attached to the frame. The resting or non-deformed shape of the device can be planar or non-planar with convex shape and generally a circular or ovoid “ring” with a central membrane, although other shapes are described herein. To deploy the retractor, the retractor is first deformed, and the deformed retractor is then fit through a surgical opening to reach a surgical area such as a body cavity. Once inside the cavity, the deformed resilient retractor is released and the frame of the retractor extends to push against the wall of the body cavity. The forces between the retractor and the wall of the body cavity make the retractor self-retaining inside the cavity while correspondingly forming a working space adjacent the retractor. The frame supports the membrane attached such that the membrane forms a barrier against relevant organs inside the cavity therefore retracts or retains the organs to create a working space inside the cavity. In general, the frame can be deformed into a non-planar convex shape inside the cavity during deployment with a concave surface facing the working space inside the cavity. Surgical procedure can then be performed inside the working space with the retained organs out of the way. The resilient retractor can be shifted around inside the body cavity to create different working spaces to facilitate the surgical process. The membrane additionally may be transparent to allow surgeon to inspect the retracted organs during surgery. After the surgery, the retractor is deformed and then removed from the body cavity through the surgical opening.

The retractor described herein provides easier access to surgical area during surgical operation by retracting non-relevant organs away from the surgical area. The retractor can be used in a variety of surgical procedures including, for example, surgery of the GI tract, urinary system, reproductive system, abdominal wall, or pelvic floor. The retractor can be used for laparoscopic surgery as well as open surgery and is particularly suited for open laparotomy, mini-laparotomy, pure laparoscopic surgery, hand assisted laparoscopic surgery (HALS), or single incision laparoscopic surgery (SILS). Comparison of different laparoscopic surgeries are discussed in by Montero et al. in Am. Surg. 2011, 77(1):73-77, entitled: “Single Incision Laparoscopic Surgery (SILS) Is Associated with Poorer Performance and Increased Surgeon Workload Compared with Standard Laparoscopy,” incorporated herein by reference. The retractor can be used in human subjects or can be adapted for veterinarian use. The retractor in general is biocompatible. The term “biocompatible” used herein refers to devices that are compatible with living cells, tissues, organs, or systems in the context of the uses described herein, and is effectively not cytotoxic or immunogenic. The biocompatible retractor in general is sterile prior to use and can be distributed within sterile packaging.

Abdomino-pelvic surgical retractors are traditionally metallic devices configured with a variety of blades, ratchets and hinges to expose the viscera of the abdomen and pelvis. More recent abdominal wall retractors have taken the form of tubular plastic sleeves, placed within the incision and retract the abdominal wall and protect the wound edges, as described in U.S. Pat. No. 5,460,170 to Hammerslag et al. entitled: “Adjustable Surgical Retractor”, incorporated herein by reference. Retractors with rollable sleeves coupling outer and inner rings are described in U.S. Patent Application No. 20080281161 to Albrecht et al. entitled: “Surgical Retractor with Gel Pad”, U.S. Patent Application No. 20080281162 to Albrecht et al. entitled: “Surgical Retractor”, and U.S. Patent Application No. 20080021362 to Fihe et. al. entitled: “Roll-up Wound Protector with Tricuspidate Ring”, all incorporated herein by reference. These wound retractors are generally concerned about protecting incisions with the rollable sleeve, which is generally cylindrical in shape with the outer ring attached at one end of the cylindrical sleeve and the inner ring attached at the other end of the cylindrical sleeve. These devices by design have limited if any ability to form a desired working space within the patient, and the present deformable retractors can be used in conjunction with these wound retractors as described further below. Also, a deformable sheet type retractor is currently marketed by Medline Industries under the name “Dash Retractor.” Dash® Retractor lacks the frame and transparent membrane provided with the improved retractors described herein such that the improved retractors herein provide significantly improved performance and versatility while still providing convenient placement and retrieval. As described herein a resilient frame of the improved retractor contacts the inner wall of the cavity within the patient to help hold the retractor in place.

One exemplary surgical procedure involves abdominal surgery. During abdominal surgery, small bowel loops can fall into the pelvis and lower abdomen if unrestrained, obscuring the surgical field. This is especially true during less invasive surgery such as HALS, SILS and other Laparoscopic procedures. The improved retractors described herein can be deployed inside the abdominal cavity and retract the small bowel out of the lower abdomen and pelvis, thus facilitating the surgery. In one embodiment, the retractor is inserted into a small laparoscopic incision by twisting the frame into a “figure-of-eight” shape and further folding the figure-of-eight upon itself to make a two layer circular configuration having about half the diameter of the original retractor. The retractor with reduced size can then be inserted into the peritoneal cavity where it “unfurls” and assumes substantially its original size and retracts small bowel out of the pelvic cavity.

The retractors described herein are applicable to other surgical procedures including laparoscopic or open sigmoid colectomy, ileocolic resection, hysterectomy, pelvic floor repair or resection fixation, or repair of the rectum or bladder. The improved deformable retractor in general can be inserted into the abdominal cavity of a patient undergoing surgery via a SILS, HALS or laparoscopic access device or even through an abdominal wall incision. Once deployed, a circular retractor assumes a generally ovoid shape within the peritoneal cavity, and is further “bent” into a convex form by pressure from the patient's lateral abdominal wall. During surgery, the retractor is held in place by means of anterior-posterior pressure exerted on the retractor by the anterior abdominal wall and posterior retroperitoneal structures. It is also held in place by lateral pressure exerted on it by the lateral abdominal wall and hence self-retaining. Having become “wedged” into the abdominal cavity, the central membrane of the retractor exerts axial pressure on the abdominal viscera and prevents bowel loops from entering the surgical field. The retractor therefore applies forces in all 3 dimensions, hence can be referred to as a 3-dimensional retractor.

The extent of compression exerted on the retractor and the eventual shape it adopts is determined by the size of the abdominal cavity, and the degree to which the peritoneal cavity is distended e.g. during laparoscopic or open abdominal surgery. The device can therefore be used both in “open” or “laparoscopic” abdominal surgery. The retractor can be placed in the abdominal cavity with a convex membrane surface facing toward the head of the patient to retract viscera in the axial plane and keep them out of the surgical field. In an alternative embodiment, the device is generally ovoid-shaped at rest, and is further compressed into an ovoid shape by anterior-posterior pressure from the anterior abdominal wall and retro-peritoneum. The natural un-deformed configuration of the retractor can be a ring or other appropriate shape of soft “springy” malleable polymer or plastic, which prevents excessive pressure on the abdomino-pelvic viscera, but effectively keeps the viscera out of the surgical field.

The frame of the retractor can be made of biocompatible metal, polymer or a combination thereof. The frame can adopt any reasonable shape and size to better fit into body cavities of various sizes. For example, the retractor may be round, square, oval, rectangular, oblong, or polyhedral with a perimeter of approximately 5 to 200 inches (12.7 to 508 cm). Additionally, the retractor may be a composite shape such as bean, figure of eight, or U-shape. The retractor in general may be planar or non-planar with a convex three dimensional shape in its natural un-deformed configuration.

The different shaped retractor offers advantage to meet a variety of surgical needs. The retractor may additionally be attached to other instruments such as laparoscopic access device or a wound retractor to provide integrated access to surgical area of interest.

The cross section through the frame, i.e., through the frame effectively perpendicular to an axis along the frame forming the general shape of the frame, may adopt various shapes and sizes to suite any particular needs also. The frame has a thin shaped member that forms a border of a central opening For example, the cross section through the frame maybe round, oval, semi-circular, triangular, or polygonal and has a perimeter of about 0.25 cm to about 10 cm. A cushion, such as with a polymeric gel or soft elastic polymer can be used to pad around the frame to prevent damage to the wall of the body cavity during self-retaining deployment.

The membrane of the retractor can be made of a biocompatible sheet, fabric, net or a combination thereof. In general, the membrane is secured around majority or all of the frame and at least a portion of the membrane generally remains attached to the frame while the retractor is being used in its retractor function. The membrane may contain perforations for access the retracted organ during the surgery. Alternatively, the membrane can be made of material that can be punctured through by surgical instruments such as a laparoscope. A range of materials can be used for the membrane as described herein, and the membrane can be relatively impermeable or the membrane can have pores, or other opening, such as an open weave, as long as the membrane functions to restrain selected organs.

### Structural Elements of the Retractor and Formation Thereof

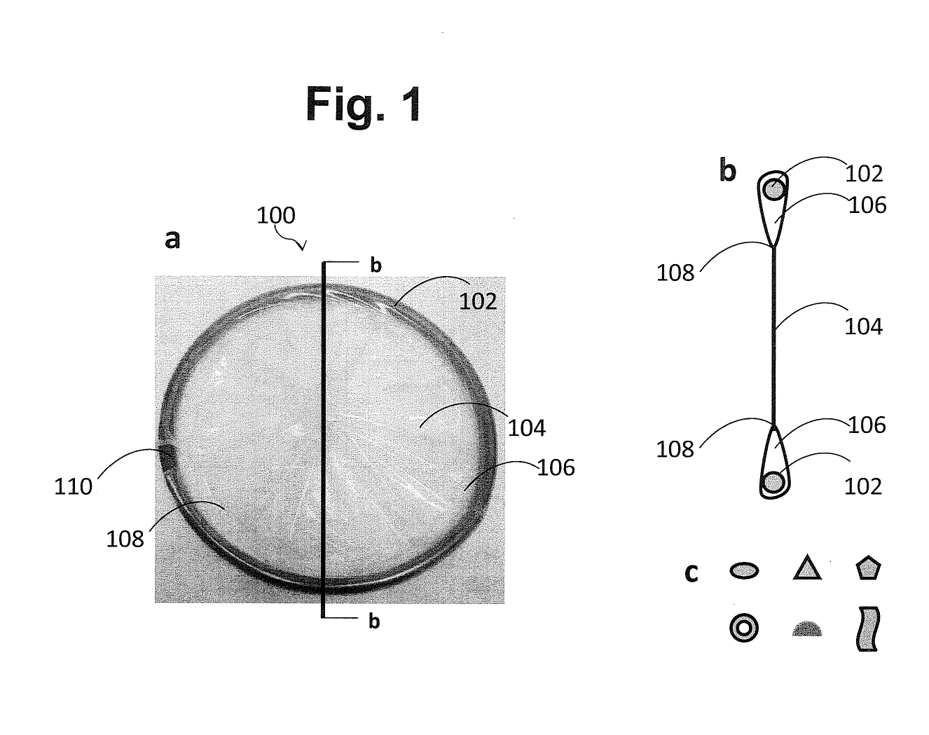

The biocompatible retractor disclosed herein generally has a deformable resilient frame with a deformable membrane secured around majority or all of the perimeter of the frame to form a generally planar surface. The frame can be constructed out of resilient metal, polymer, or a combination thereof. The membrane may be a plastic sheet or the like, which serves as the retractor surface in the axial plane to retract for example viscera out of the pelvis. It can be desirable for the membrane to be transparent. A photograph of an embodiment of the retractor with a circular frame and a transparent plastic sheeting is shown in FIG. 1. Specifically, retractor 100 comprises a frame 102 and a membrane 104 wrapped around the majority of the frame to secure the membrane, as shown in FIG. 1a. The wrapped area 106 of the membrane 104 has sealing lines 108 securing the membrane 104 around the frame 102. For example, sealing lines can correspond with locations of heat bonding and/or adhesive bonding of the wrapped portion of the membrane to secure the membrane to the frame. Frame 102 has a relatively thin member that forms a central opening across which the membrane extends. The retractor can optionally have a segment 110 of the frame that does not have membrane attachment, leaving expansion room for the membrane during for example the deformation process of the retractor, although the membrane can be attached completely around the frame in some embodiments.

In general, the frame can form a closed structure around the central opening or an open structure that surrounds a majority or all of the perimeter of the central opening. The frame can be planar in its relaxed position, or the relaxed position of the frame can be a non-planar convex shape. The perimeter formed by the frame provides for attachment of the membrane across the central opening or a suitable portion thereof. As noted above, the membrane may or may not be attached at all points of the border around the central opening formed by the frame. In some embodiments, the membrane is attached at a majority of the border points.

A cross sectional view through the retractor of FIG. 1a along the b-b line is illustrated in FIG. 1b showing the wrapped around area 106 secured by sealing lines 108 around the frame 102, which has a circular or round cross section. The frame may have alternative cross section shapes through the frame element such as triangular, oval, pentagonal, wavy rectangular, semi-circular, or circular with hollow, as shown respectively in FIG. 1c. In general, any reasonable cross sectional shape of the frame can be used. Triangular and wavy rectangular frame in particular may provide better self-retaining properties for the retractor once deployed as the triangular or wavy rectangular frame may provide more latch when in contact with wall of the body cavity. Additionally, in the embodiments where the cross sectional shape of the frame is semi-circular or triangular, twisting and folding the semi-circular or triangular frame into a two layer configuration results in a circular or diamond cross sectional profile, which further assists in inserting the retractor into the abdominal incision.

The cross section of the frame with a circular cross section can measure from about 0.1 cm to about 3 cm in diameter, in some embodiments from about 0.25 to about 2.5 cm, and in additional embodiments about 0.5 cm to about 2 cm in diameter. For frames with alternative cross sectional shapes, the size of the cross section is more easily defined with the perimeter instead of diameter. In general, the frame can have a perimeter around a cross section of the frame of about 0.25 cm to about 10 cm, in some embodiments of about 0.5 cm to about 8 cm, and in additional embodiments of about 1 cm to about 6 cm. A person of ordinary skill in the art will recognize that additional ranges of diameters and perimeters within the explicit ranges above are contemplated and are within the present disclosure.

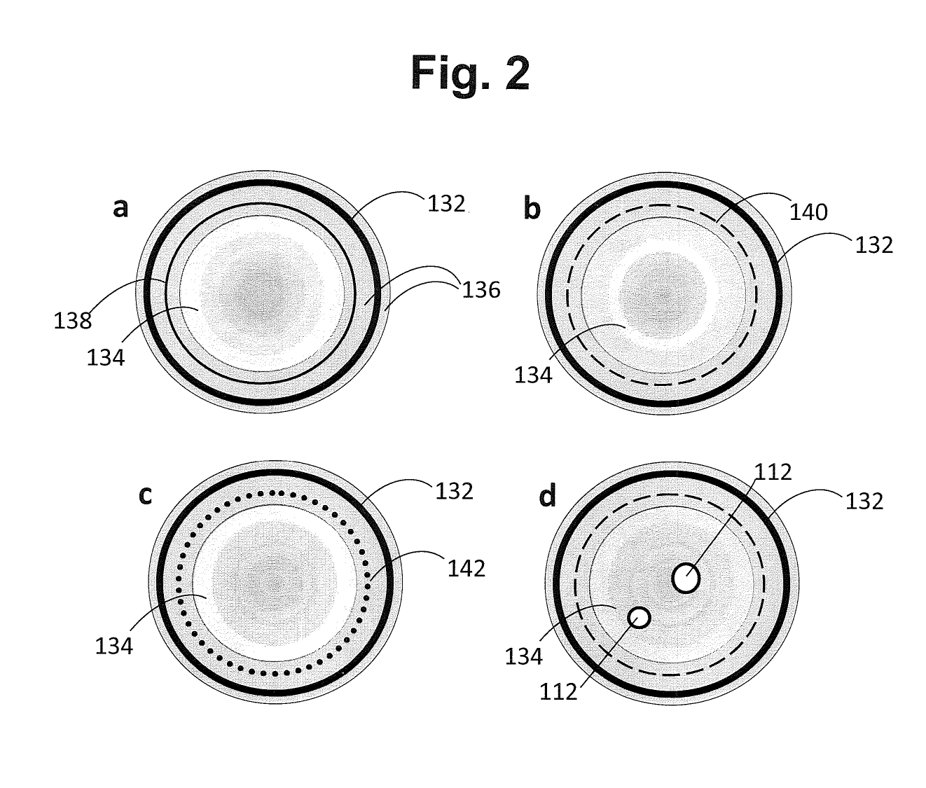

The membrane may be secured directly to the frame using an adhesive or heat sealing techniques, although other techniques known in the art may also be used. Alternatively, the membrane may wrap around the frame and attached or sealed to itself as shown in FIGS. 1a and 1b. The wrap around configuration with various sealing methods is further illustrated by schematic diagrams in FIGS. 2a-2c. Referring to FIG. 2a, membrane 134 wraps around frame 132 creating a wrapped around area 136 around the frame that is secured by sealing line 138, which is the point of attachment of the membrane onto itself via adhesive or heating of the membrane material. Alternatively, the sealing line may be created with stitches 140 as shown in FIG. 2b or with staples 142 as shown in FIG. 2c. While these figures show the membrane attached to itself after wrapping around the frame, in additional or alternative embodiments, the membrane can be directly attached to the material of the frame. Thus, the membrane material can be glued, heat bonded, stitched and/or stapled directly to the frame. In general, the membrane can be attached around a majority of the perimeter of the frame, although the membrane may be attached around the entire perimeter of the frame.

The frame of the retractor in general is composed of resilient, flexible material to permit twisting, bending, and generally deformation of the retractor. Padding or cushion can be added around the frame to improve traction between the retractor and the wall of the body cavity while preventing applying excess pressure to cause injury of the tissue of cavity.

In general, the frame can be formed from one or more biocompatible materials, including, for example, metals, such as stainless steel or alloys, e.g., Nitinol®, or polymers such as polyether-amide block co-polymer (PEBAX®), nylon (polyamides), polyolefins, polyesters, polycarbonates or other suitable biocompatible polymers including elastomeric polymer, such as suitable polyurethanes, polydimethyl siloxane, acrylonitrile butadiene styrene (ABS), high density polyethylene (HDPE), rubber, polyisoprene (i.e., synthetic rubbers), and polytetrafluoroethylene. Spring metals or shape memory metals, such as Nitinol®, can be particularly useful. A metal frame can be covered in a polymeric cover or the like. In some embodiments, different portions of frame can be formed from different materials to introduce desired stiffness/flexibility for the particular portion of the frame. In some embodiments, the frame can be made from polymer embedded with metal wire. Suitable polymers include, for example, polyamides, i.e., nylons. The metal wire can be braided, coiled or otherwise placed over a polymer tubing liner with some tension. A polymer jacket can be then placed over the top of metal wire or the like. Upon heating over the softening temperature of the polymer and subsequent cooling, the wire becomes embedded within the polymer. The liner and jacket can be the same or different materials. The wire adds additional mechanical strength while maintaining appropriate amounts of flexibility. In general, the frame is made of material that has sufficient resilience to substantially regain its resting configuration after being deformed, which is self-extendable. The frame may be a durometer from about 40 A to about 90 A on the A durometer scale or from about 50 D to about 90 D on the D durometer scale, as specified in ASTM protocol D2240-00. Frames formed from elastic polymers, such as rubbers or the like, can be convenient and relatively inexpensive.

The membrane can be constructed from a biocompatible sheet, fabric, net, or a combination thereof. The membrane may be made out of transparent or semi-transparent materials, such as polymers. In some embodiments, it may be desirable to have perforation on the membrane for the purpose of passing surgical instruments such as laparoscope through the membrane. FIG. 2d for examples shows a schematic diagram of a round shaped retractor with perforations 112 on the membrane. The membrane can alternatively be constructed from material that can be punctured by medical instruments such as a laparoscope to gain better visualization from trochar sites proximal to the retractor.

The membrane may be clear, transparent, translucent, opaque and made of a variety of different materials with different characteristics. In one embodiment, the membrane can be made from an elastomer such as polyisoprene, polyurethane, silicone polyurethane, or silicone. In some embodiments, the membrane may be non-elastic or only slightly elastic so that the membrane can restrain organs or other body portions without significantly distending the membrane. Thus, inelastic materials such as polyethylene terephthalate can be used to form the membrane or portions thereof. The thickness of the membrane may vary, for example, from about 0.005 inches (0.0127 cm) to about 0.1 inches (0.254 cm) and in some embodiments of about 0.01 inches (0.0254 cm) to about 0.05 inches (0.127 cm). A person of ordinary skill in the art will recognize that additional ranges of thickness within the explicit ranges above are contemplated and are within the present disclosure.

The membrane in general is foldable and has sufficient strength to retain organ while being extended around the frame. The retractor can be made of disposable materials, such that the retractor is thrown away after use, although the retractor can be made of reusable material in some embodiments.

The resilient nature of the frame permits twisting and folding of the retractor to allow insertion into small abdominal wall incisions, and permits the retractor to conform to the inner contours of the inner aspect of the patient's abdomen to effectively retract bowel away from the surgical field. Clinical experience has demonstrated that during open abdominal laparotomy in an approximately 70 kg male the abdominal cavity measures approximately 6-8 inches in anterior to posterior (A-P) plane and 10-12 inches in lateral plane. Thus, for a circular-shaped retractor for a majority of patients, the natural or “resting” dimensions of the retractor can have a suitable diameter of approximately 6 to 20 inches, in some embodiments from about 8 to about 18 in and in additional embodiments from about 10 to about 16 inches in diameter. For a generally convex-shaped retractor used for open surgeries, the resting A-P dimensions within the patient can be from about 2 to about 12 inches, in some embodiments from about 4 to about 10 inches and in further embodiments from about 6 to about 8 inches. Lateral dimensions, across the patient, of a generally non-planar convex shaped retractor within the patient can be about 6 to about 20 inches, in further embodiments from about 8 to about 18 inches and in other embodiments form about 10 to about 16 inches. A person of ordinary skill in the art will recognize that additional dimensional ranges within the explicit ranges above are contemplated and are within the present disclosure.

Furthermore, clinical experience has demonstrated that during laparoscopic surgery in an approximately 70 kg male, A-P peritoneal dimensions are 9-12 inches and lateral dimensions are 10-12 inches. The lateral dimensions are similar to open cases, since laparoscopic insufflations of the abdomen increases the A-P dimension much more than the lateral dimensions, which stays reasonably unchanged. For a circular retractor, the natural or “resting” dimensions of the retractor has a diameter from about 6 to about 24 inches, in further embodiments from about 8 to about 20 and in other embodiments from about 10 to about 16 inches. For a generally convex-shaped retractor within the patient used for laparoscopic cases, the resting A-P dimensions can be in some embodiment from about 6 to about 20 inches, in further embodiments from about 8 to about 18 inches and in other embodiment from about 9 to about 14 inches. Lateral dimensions for a generally ovoid retractor within the patient would be approximately the same as for an “open” version of the retractor i.e. from about 6 to about 20 inches, in further embodiments from about 8 to about 18 inches and in additional embodiments from about 10 to about 16 inches. A person of ordinary skill in the art will recognize that additional ranges of dimensions for non-circular embodiments within the explicit ranges above are contemplated and are within the present disclosure.

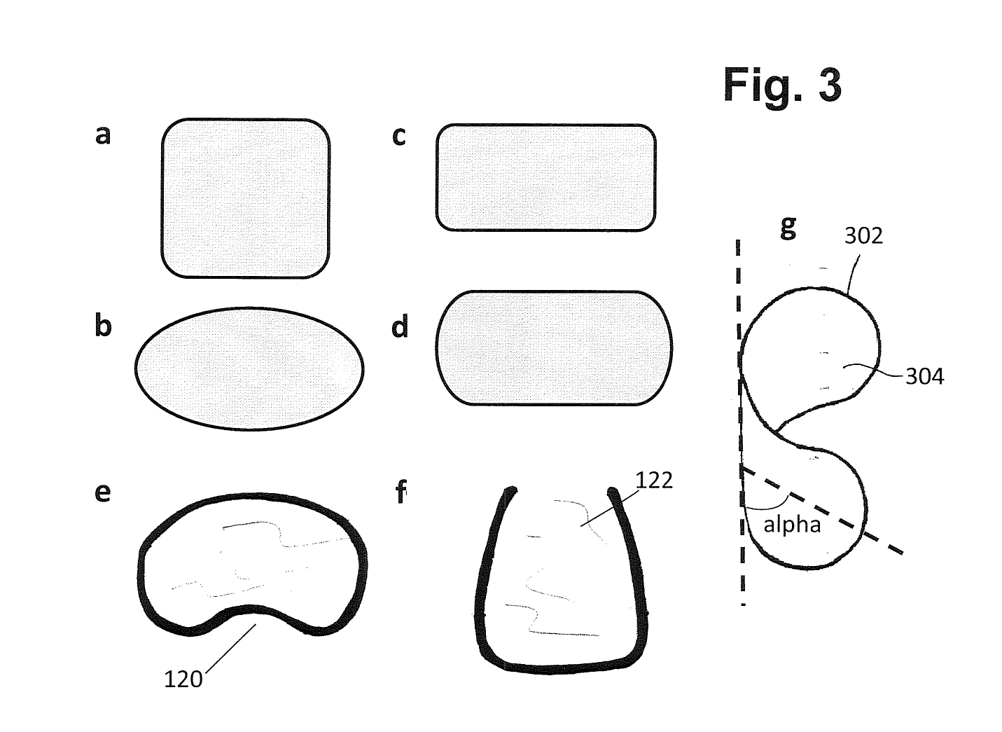

Besides round or circular shape, alternative planar retractor shapes such as square, rectangle, oval, oblong, bean, and “U” or horse shoe or composite shape shown in FIG. 3a-f, can be adopted. Some of these frame shapes form a closed structure around the interior. The frame of the “U” or horse shoe shaped retractor does not surround the entire circumference of the membrane and has an opening area 122 that can be more easily deformed than other parts of the retractor. Additionally, retractor can be made with a frame that is non-planar in its resting or natural configuration. An example of a non-planar convex circular retractor is shown in FIG. 3g, although retractors with other shapes can be made three dimensionally convex also. The degree of curvature out of the plane can be selected to suite desired properties of the retractor. The non-planar convex shaped retractor may be desirable in some applications, as the retractor may or may not rely on the interaction between the wall and the frame only to create the convex shape for restraining the organs within the patient. The dimensions of the frame of a non-planar retractor can be evaluated by distorting the frame to a planar configuration for the measurements, and then the measurements described above for the naturally planar retractors can be applied to the non-planar designs.

In general, the retractor can be made of various sizes and configurations for use in various size humans and animals depending on the size and weight of the individual (e.g. small, medium and large), body habitus, prior abdominal surgery, size and dimensions of the peritoneal cavity, and whether open vs. laparoscopic surgery is being performed. For example, when deployed inside a body cavity, the concavity of a bean shaped retractor may be placed over retroperitoneal vessel and/or conduit to prevent compression or occlusion of the vessel and/or conduit. For devices of alternative shapes, the size of the retractor is more conveniently defined with the perimeter instead of diameter. In general, the retractor can have a perimeter from about 5 to about 200 inches (12.7 to 508 cm), in some embodiments from about 10 to about 150 inches (25.4 to 381 cm), in additional embodiments from about 20 to about 100 inches (50.8 to 254 cm). A person of ordinary skill in the art will recognize that additional ranges of perimeter within the explicit ranges above are contemplated and are within the present disclosure.

The retractor may additionally be combined with other surgical instruments such as a wound retractor or a laparoscopic access device. In one embodiment, the retractor may affix to the proximal aspect of an abdominal wound retractor such as the “Alexis” retractor by Applied Medical, hand-assisted laparoscopic access devices such as the “Gelport” also from Applied Medical, or the “Dextrus” hand access device from Ethicon Endosurgery. The wound retractor and the access devices commonly has an inner plastic or polymer ring that partially retains the retractor in the abdominal wound. The retractor described herein may attach to a selected location of the ring of the wound access device using an approximately clip on attachment which attaches the retractor to the inner ring of the abdominal wound retractor. Such a clip on attachment can attach around the inner ring, while not interfering impinging or perforating the sleeve aspect of the abdominal wound retractor and retracts viscera into the upper abdomen. Various fasteners can be adapted for interfacing the surgical retractor described herein with the wound access retractor lower ring. The interfacing of the surgical retractor with the wound retractor provides for ability to obtain the advantages of both devices while limiting any interference between the devices.

The retractor described herein can be made to be disposable items to avoid the need for subsequent cleaning and sterilization. The generally sterile biocompatible retractor may additionally be coated with bacterialcidal or anti-inflammatory agents to further prevent infection or other complication during the surgical procedure. In some embodiments, it may be advantageous to make the retractor impermeable to fluids to provide a relatively dry environment in the working space.

### Retractor in Surgical Applications

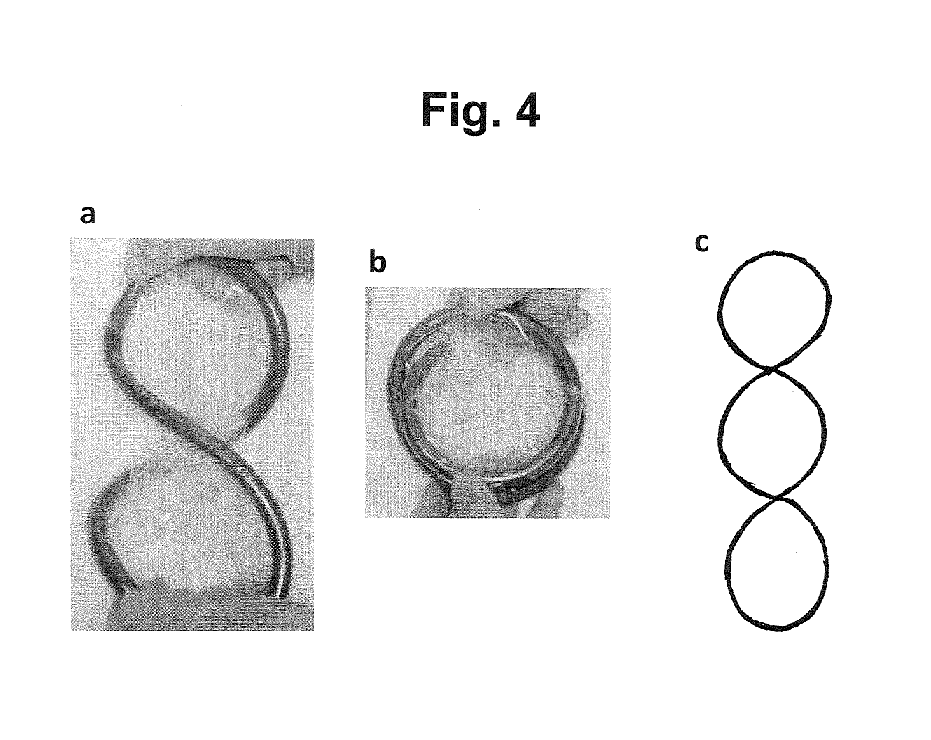

A circular retractor can be used in abdominal surgeries by twisting the retractor into a folded shape, such as a figure-eight shape or other convenient folded shape. For example, a figure eight shape formed form a circular retractor can be further folded upon itself to make it half its original diameter. Non-circular shaped retractors can be similarly appropriately folded for insertion into the patient. The folded or deformed retractor can then be inserted into a small laparoscopic incision into the peritoneal cavity. Having inserted the retractor into the peritoneal cavity, the retractor “unfurls” and assumes substantially its original size and retracts small bowel out of the pelvic cavity. As shown in FIG. 4a, a circular retractor 100 of FIG. 1a is folded into a figure-of eight shape, which when folded upon itself forms a two layered circular structure with about half of its original diameter as shown in FIG. 4b. When released, the deformed retractor can substantially regain its resting configuration, and the particular resulting shape and size of the deformable retractor can conform to the peritoneal cavity into which it is placed.

The resilient retractor when deformed can be inserted into smaller incisions than would otherwise be possible. The unfolding of the deformed retractor to assume substantially its original size inside a body cavity allows the retractor to push against the wall of the cavity to become self-retainable inside the cavity. The resiliency is generally not excessive so that the retractor does not injure any organs or the wall of the body cavity.

Specifically, for use in the abdominal cavity, the retractor can be compressed in the anterior-posterior (A-P) plane by pressure from the anterior abdominal wall into an ovoid shape, and further bent in the lateral plane into a convex or non-planar “U” form, by pressure from the lateral abdominal wall. Although convex deployed configuration is discussed, it is understood that other deployed configurations such as planar or concave relative to the lower part of the patient are also contemplated and within the present disclosure. The resulting two dimensional pressures not only distribute the pressures substantially along the entire circumference of the retractor to reduce or prevent organ injury or excessive local pressure at any one location, but also result sum of the individual force vectors maintain the retractor in place without additional external force or devices. In addition, as the retractor tries to assume its original configuration and conforms to the inner contours of the abdominal cavity, the central membrane expands in an anterior-posterior and lateral direction to serve as the retractor surface in the axial plane, which effectively occludes the abdominal cavity, retracts viscera out of the pelvis, and thereby prevents the small bowel from entering the surgical field. The retractor provides retraction in all three planes and is self retaining. In some embodiments, it may be desirable to use a retractor that has a natural non-planar convex shape in the un-deformed or resting configuration. When such a non-planar convex shaped retractor is released inside a body cavity, it regains substantially its convexity and pushes organs away to create desired working space.

By avoiding significant pressure being applied to the retroperitoneum and by the distribution of pressure around the entire periphery of the retractor, the great vessels in the retroperitoneum (vena cava and aorta) are not significantly impinged, compressed or obstructed. This use of the device avoids arterial insufficiency (which results from aortic compression) and also avoids impeding venous return (which results from vena caval compression), both of which can be potentially life threatening. In some embodiments, the deployed circular retractor or other suitable device design avoids trauma to or compression of the retroperitoneal structures during sudden deflation of the abdomen, which can happen from time to time during laparoscopic surgery. In this event, the retractor simply distorts to a more ovoid shape exerting little or no additional force on the retroperitoneum or viscera. Additionally, the retractor avoids pressure necrosis or even perforation of small bowel or other delicate viscera. The reduction or elimination of compression or occlusion of the major blood vessels avoids a potential drop in blood pressure, which can be a life-threatening problem with metal mechanical retractors known in the art. In a further embodiment, the retractor may be bean shaped to provide the concavity of the bean over the retroperitoneum to further reduce compression of the great vessels.

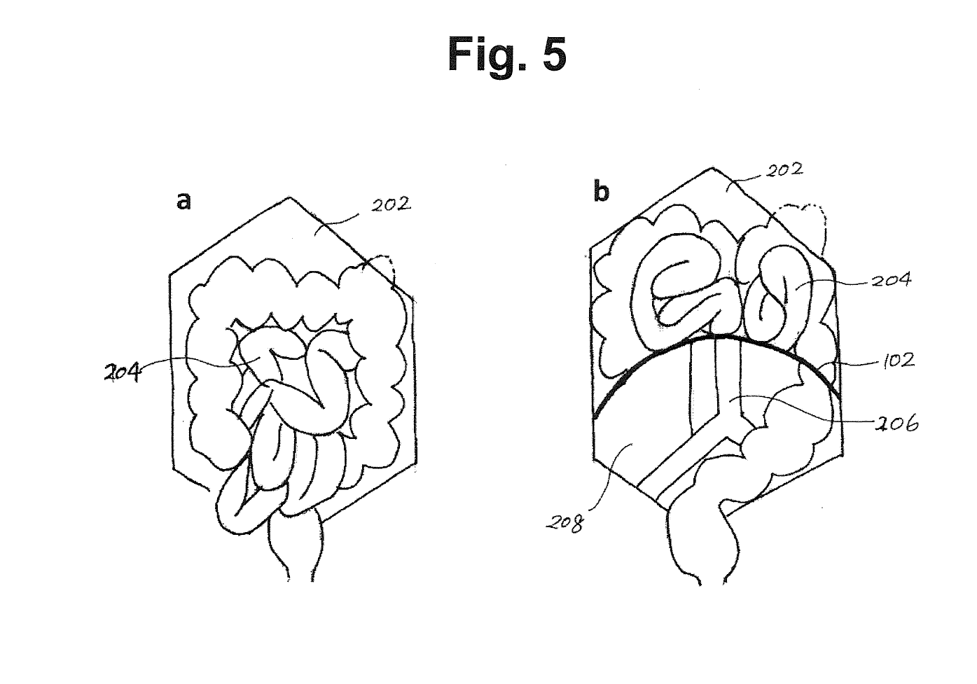

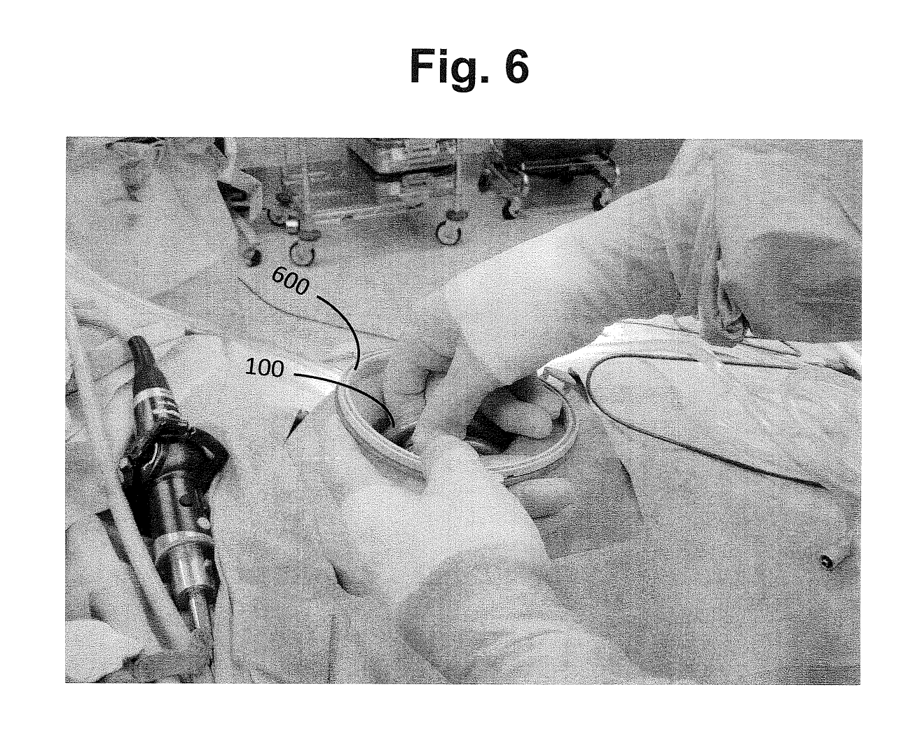

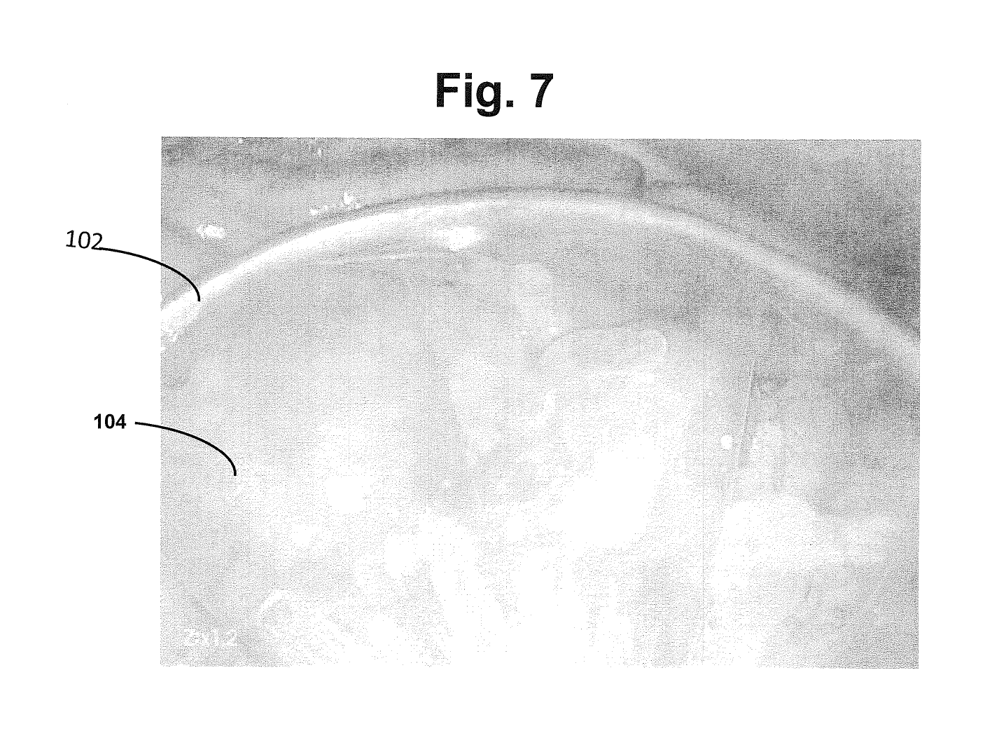

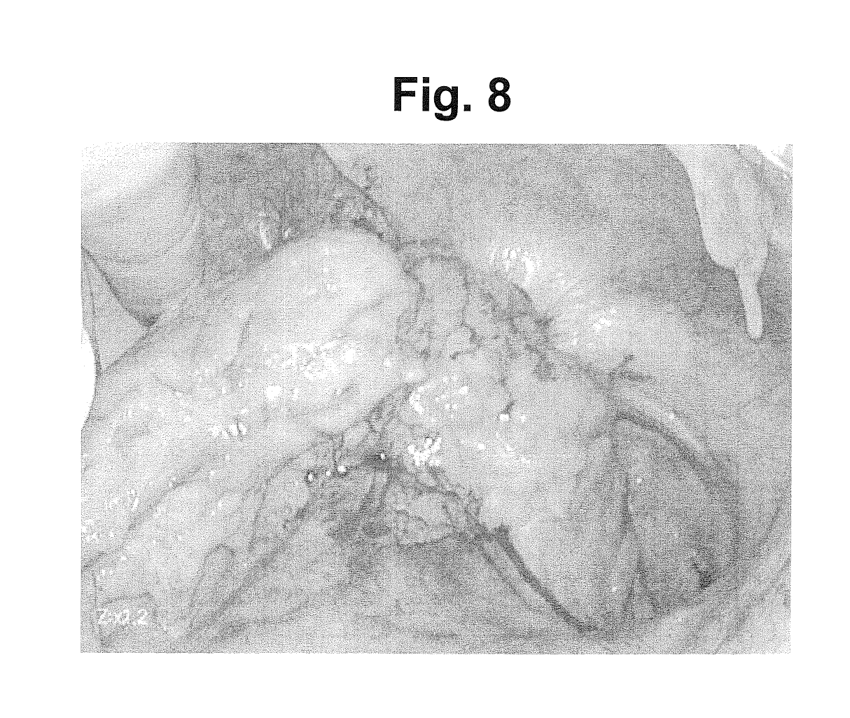

Referring to FIG. 5a, a schematic diagram of abdominal cavity 202 is shown with bowel loops 204 inside the pelvic cavity. A circular retractor placed inside the abdominal cavity is shown in FIG. 5b, whereupon it gently “unfurls” to assume substantially its original size. As a result of the resilient frame 102, the retractor assumes the contours of the inner aspect of the patients inner abdominal wall and forms a convex shape to effectively retract bowel loops into the upper abdominal cavity and prevent them from falling into pelvic cavity where surgery can be performed. As a result of the contraction, a working space 208 is created inside the pelvic cavity exposing great vessels 206. After completion of the surgery, the retractor is gently pulled out of the incision, and its flexible nature avoids any injury to the abdominal viscera or the incision itself. FIGS. 6-8 are photos of the retractor of FIGS. 4a and 4b being deployed inside a body cavity during surgery and creating a working space free from the interference of the retained organs. Specifically, FIG. 6 is a photograph of the folded retractor being inserted through a wound access device 600 into abdominal cavity during a laparoscopic surgery. Once inside the abdominal cavity, the retractor is released and effectively retain organ as shown in FIG. 7 where retained organ can be seen through the transparent membrane of the retractor. The working space created by the retractor substantially free from the retained organs is shown in FIG. 8, thus providing improved access to surgical site of interest.

In some embodiments, the 3-D retractor can be rotated around the peritoneal cavity or around the inner ring of the abdominal wound retractor to provide access to the left colon or splenic flexure for the purpose of surgical mobilization, whilst still retracting the viscera out of the surgical field. For this purpose, the retractor is rotated counter-clockwise within the peritoneal cavity. The retractor may also be rotated or repositioned to enable mobilization of the right colon and hepatic flexure, again keeping the viscera out of the surgical field. For this purpose, the retractor is rotated clockwise within the peritoneal cavity.

In some embodiments, the lateral one thirds of a circular resting retractor may be individually “twisted” 180 degrees in their axial plane, preferably one in a clockwise and the other in a counter-clockwise manner This forms a symmetrical shape of three serial ovoids: A central ovoid, and two lateral ovoids as shown in FIG. 4c. When each lateral ovoids are released, they untwist in the opposite directions, when viewed end-on from one direction. Since both lateral ovoids are located at diametrically opposing ends of the central ovoid, the lateral ovoids “untwist” in a mirror image manner of each other. The end result of this is that the retractor untwists in a symmetrical manner when viewed side-on, to providing a symmetrical and effective retractor capable of retracting bowel out of the pelvis. Having formed both lateral ovoids, each lateral ovoid may then be folded 180 degrees to overlay the central ovoid, resulting in a 3-layer, generally circular or ovoid shape, one third of the original diameter. When the 3-layer deformed retractor is inserted into peritoneal cavity, the two lateral one third “ovoids” unfurl laterally and untwist axially to assume the generally U shaped retractor configuration detailed above. Of course, for a particular use and for particular device configuration if not circular, the particular folding of the surgical retractor can be selected appropriately by the practitioner based on the teachings herein. The deployed retractor may also adopt a concave shape directing the bowl or other organs toward the head, or a planar shape inside the body cavity, which can be particularly effective for embodiments of membranes that are essentially not elastic.

The aforementioned twisting and folding maneuver can be repeated a plurality of times, to further decrease the diameter of the retractor for example to one quarter, one eighth, or one sixteenth etc of the original resting configuration. This smaller configuration of the retractor facilitates insertion of the retractor into the peritoneal cavity via an abdominal wound retractor or hand assisted laparoscopic access retractor. For example, the retractor can be used in conjunction with a SILS access device by Covidien or a plastic HALS retractor such as the “Dextrus” by Ethicon Endosurgery, or with the “Lexus” HALS retractor made by Applied Medical. This combination provides much improved surgical exposure and is highly desirable during laparoscopic surgeries.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What is claimed is:

1. A biocompatible deformable retractor comprising,
a deformable resilient frame comprising a border around a majority or all of a perimeter around a central opening, and a deformable membrane secured around at least a portion of the border to form a restraining structure across the central opening,
wherein the retractor is approximately planar or a non-planar convex shape in its natural un-deformed configuration.

2. The retractor of claim 1 wherein the frame has a cross section through the frame that is round, oval, semi-circular, triangular, or polygonal having a perimeter of about 0.25 cm to about 10 cm.

3. The retractor of claim 1 further comprising a cushion around the frame.

4. The retractor of claim 1 wherein the membrane is transparent.

5. The retractor of claim 1 further comprising perforation through the membrane.

6. The retractor of claim 1 wherein the retractor is about round, square, oval, rectangular, oblong, or polyhedral shape.

7. The retractor of claim 1 wherein the retractor is bean, figure of eight, or U-shape.

8. The retractor of claim 1 wherein the retractor in its natural un-deformed configuration has a perimeter of approximately 5 to 200 inches (12.7 to 508 cm).

9. The retractor of claim 1 wherein the retractor is self-extendable.

10. The retractor of claim 1 wherein the membrane comprises a biocompatible sheet, fabric, web, or a combination thereof and the frame comprises polymer, metal, or a combination thereof.

11. A method of using a biocompatible retractor to retract one or more organs in a body cavity to form a working space in a living subject, the method comprising,
deploying a retractor by deforming the retractor, inserting the deformed retractor into the body cavity through a surgical opening, and releasing the retractor in the body cavity to retract the organ in the cavity to form the working space,
performing a surgical procedure in the working space, and
removing the retractor from the body cavity through the surgical opening,
wherein the retractor comprises a deformable resilient frame and a membrane attached to the frame, and
wherein the retractor is self-retaining inside the cavity with the frame pushes against the wall of the cavity while the membrane presses against the organ to form the working space inside the cavity.

12. The method of claim 11 wherein the membrane remains attached to the frame throughout the procedure.

13. The method of claim 11 wherein the surgical procedure is a laparoscopic surgery.

14. The method claim 11 wherein the surgical procedure is a surgery of the GI tract, urinary system, reproductive system, abdominal wall, or pelvic floor.

15. The method claim 11 wherein the surgical procedure is open laparotomy, mini-laparotomy, pure laparoscopic surgery, hand assisted laparoscopic surgery, or single incision laparoscopic surgery.

16. The method of claim 11 wherein the organ is GI tract, the body cavity is peritoneal cavity, and the working space is pelvic cavity.

17. The method of claim 11 wherein the retractor deployed inside the cavity has a convex shape pushing against the organ to make the working space.

18. The method of claim 11 wherein the retractor is bean shaped and when deployed the concavity of the bean shape is placed over retroperitoneal vessel and/or conduit to prevent compression or occlusion of the vessel and/or conduit.

19. The method of claim 11 further comprising repositioning of the retractor inside the cavity to make another working space.

20. The method of claim 11 further comprising puncturing the membrane by laparoscopic instruments or laparoscopes.

21. A method of making a biocompatible deformable resilient retractor, comprising,
securing a membrane to a deformable resilient frame to form the retractor that is approximately planar or a non-planar convex shape in its natural un-deformed configuration,
wherein the frame comprises a border around a majority or all of the perimeter of a central opening and the membrane is secured around at least a portion of the border to form a restraining structure within the central opening.

22. The method of claim 21 wherein the membrane comprises a biocompatible sheet, fabric, web, or a combination thereof and the frame comprises polymer, metal, or a combination thereof.

23. The method of claim 21 further comprising attaching the retractor to a wound retractor or a laparoscopic access device.
